(19) United States
(12) Patent Application Publication
(10) Pub. No.: US 2024/0209304 A1
(43) Pub. Date: Jun. 27, 2024

(54) DEVICE, METHOD AND APPARATUS FOR CONTROLLING HUMIDITY IN AN INCUBATOR

(71) Applicant: THRIVE BIOSCIENCE, INC., Wakefield, MA (US)

(72) Inventor: Alan BLANCHARD, Wakefield, MA (US)

(73) Assignee: THRIVE BIOSCIENCE, INC., Wakefield, MA (US)

(21) Appl. No.: 18/288,496

(22) PCT Filed: Apr. 26, 2022
(86) PCT No.: PCT/US2022/026275

§ 371 Date: Oct. 26, 2023

Related U.S. Application Data
(60) Provisional application No. 63/179,636, filed on Apr. 26, 2021.

(51) Int. Cl. C12M 1/00; C12M 1/12

(52) U.S. Cl. CPC C12M 41/14; C12M 37/00

(57) ABSTRACT

A device, method and apparatus for controlling humidity in a cell incubator. The device includes a thermally conductive block having a water exuding surface, an electrical heater attached to the block and receptive of electrical energy to heat the block, at least one fan for blowing gas to be humidified over the surface of the block, at least humidity sensor attached at least downwind of the block, a controllable pump connectable to a source of water for supplying water to the exuding surface of the block and a controller receptive of output signals from the at least one humidity sensor and connected to the pump for controlling the pump based upon the output signals from the at least one humidity sensor to obtain a desired level of humidity.

### PRIORITY CLAIM

This application claims priority of U.S. Provisional Application Ser. No. 63/179,636 filed Apr. 26, 2021, the entire contents of which are hereby incorporated herein by reference.

### FIELD OF THE INVENTION

The present invention relates to cell incubators and in particular to controlling humidity in an incubator.

### BACKGROUND

Cell culture incubators are used to grow and maintain cells from cell culture, which is the process by which cells are grown under controlled conditions. Cell culture vessels containing cells are stored within the incubator, which maintains conditions such as temperature and gas mixture that are suitable for cell growth. Cell imagers take images of individual or groups of cells for cell analysis.

During the culturing of live cells it is important to control the ambient humidity in order to minimize evaporation of the aqueous nutrient medium in which they grow. Any evaporation of the nutrient medium will result in changes of the concentration of the constituents of the nutrient medium leading to unwanted changes in the cells.

One common method to achieve a high relative humidity in an incubator is to simply place a large tray of water in the incubator with the cells. Other methods include spraying a water mist into the incubator or bubbling the gas in the incubator through a container of water. All these methods result in a high but uncontrolled relative humidity.

If the relative humidity is too high, then water may condense on cool surfaces in the incubator. If a mist is used, then it may not completely evaporate, leaving droplets of water to stick and accumulate on surfaces. Liquid water, either in the tray or condensed in the incubator are prime venues for growth of bacteria and fungi and is thus best to be avoided.

The amount of energy required to evaporate water is 2260 Joules per gram. Typically, this energy comes from cooling down the surroundings, a phenomenon experienced by anyone who has worn wet clothing. This both limits the rate of evaporation and leads to cool areas of the incubator. Spraying a mist or bubbling gas through water lowers the temperature of the gas, which then has to be reheated to a desired temperature.

### SUMMARY

The object of the present invention is to improve the control of humidity in an incubator and in incubators that include imaging apparatus for imaging cell cultures during culturing.

In some embodiments, the present invention comprises a thermally conductive block, such as an aluminum block, covered with a wick, such as a piece of cotton cloth. The block is attached to an electrical heater and one or more temperature sensors. A fan blows the gas to be humidified over the cloth covered block and temperature and humidity sensors are placed both upwind and downwind of the block. A pump, capable of delivering metered amounts of water, is connected via tubing between a reservoir of water and the block such that water pumped from the reservoir will wet the cloth. Water is pumped to the block under computer control, according to measurements from the temperature and humidity sensors. Thus, the humidity can be set to any desired level.

It is desired that the amount of water pumped onto the cloth wick is not so much as to allow drops to fall or be blown off the wick. In some embodiments this can be controlled by noting the temperature of the block and the amount of power delivered to the heater to maintain that temperature. If the wick is wet, the heater's power will be expended turning water to vapor and the temperature of the block won't rise as much or may fall. If the wick is dry, the power will go into raising the temperature of the block. Thus, by monitoring the heater power and block temperature, the wetness of the wick can be evaluated and the supply of water to the wick can be controlled so it doesn't get overly wet and drip. By supplying the energy required to vaporize the water directly to the water, it doesn't have to come from the gas and the gas temperature remains constant.

It is important that the humidification system not introduce contaminants, especially viruses, bacteria, and fungi, into the incubator. To that end, in some embodiments, the water going to the block can be filtered through a 0.2 μm filter to remove bacteria and fungi. Viruses can be eliminated by exposing the water to ultraviolet light as it travels from the pump to the block.

If it is desired to sterilize the block, in some embodiments, the water pump can be turned off until the block is dry, then the fan can be turned off and sufficient power can be applied to the block to raise its temperature sufficiently to sterilize it.

In some embodiments, the water reservoir can be located outside the incubator so that the amount of water therein can be monitored, either manually or by computer, and the reservoir refilled without opening the incubator. This is an advantage over the standard method of simply placing a tray of water in the incubator.

In some embodiments, the cotton cloth wick could be any wettable fabric, comprised of natural and/or synthetic fibers. In some embodiments, it could also be a foam-like structure like a sponge. In some embodiments, instead of a wick, small grooves could be machined into the block to retain water via capillary action. Alternately, the entire block could be machined out a foam-like structure comprised of a material with suitable thermal conductivity and wettability, thus obviating the need for a wick.

In some embodiments, the incubator can include an imaging system and method of imaging. An imaging system and method of this type is described in U.S. application Ser. No. 15/563,375 filed on Mar. 31, 2016 and the disclosure of which in its entirety is hereby incorporated by reference and in PCT/US21/19757 filed on Feb. 26, 2021 and the disclosure of which in its entirety is hereby incorporated by reference.

These and other objects are achieved in accordance with the invention by a device insertable into an interior of an incubator for controlling humidity in the incubator, comprising a thermally conductive block having a water exuding surface, an electrical heater attached to the block and receptive of electrical energy to heat the block, at least one fan for blowing gas to be humidified over the surface of the block, at least humidity sensor attached at least downwind of the block, a controllable pump connectable to a source of water for supplying water to the exuding surface of the block and a controller receptive of output signals from the at least one humidity sensor and connected to the pump for controlling the pump based upon the output signals from the at least one humidity sensor to obtain a desired level of humidity.

The water exuding surface in some embodiments is wicking material attached to the block, grooves in a surface of the block, and/or a foam outer surface of the block.

The device in some embodiments includes a filter between the source of water and the block and/or a source of UV light irradiating water entering the block.

The device in some embodiments includes at least one temperature sensor at the block for sensing the temperature of the block and wherein the controller is receptive of electrical signals from the at least one temperature sensor to maintain a desired temperature of the block to prevent water from dripping from the exuding surface.

The device in some embodiments further comprising at least one humidity sensor upstream of the block and supplying a signal to the controller.

Also in accordance with the present invention is an apparatus for controlling humidity in a cell incubator comprising a cell incubator having an interior, a thermally conductive block having a water exuding surface disposed in the interior of the incubator, an electrical heater attached to the block and receptive of electrical energy to heat the block, at least one fan for blowing gas to be humidified over the surface of the block, at least humidity sensor attached at least downwind of the block, a controllable pump connectable to a source of water for supplying water to the exuding surface of the block and a controller receptive of output signals from the at least one humidity sensor and connected to the pump for controlling the pump based upon the output signals from the at least one humidity sensor to obtain a desired level of humidity.

In some embodiments, the source of water is a water reservoir disposed exteriorly of the interior of the incubator.

The water exuding surface in some embodiments is wicking material attached to the block, grooves in a surface of the block, and/or a foam outer surface of the block.

The apparatus in some embodiments includes a filter between the source of water and the block and/or a source of UV light irradiating water entering the block.

The apparatus in some embodiments includes at least one temperature sensor at the block for sensing the temperature of the block and wherein the controller is receptive of electrical signals from the at least one temperature sensor to maintain a desired temperature of the block to prevent water from dripping from the exuding surface.

The apparatus in some embodiments further comprising at least one humidity sensor upstream of the block and supplying a signal to the controller.

Further in accordance with the invention is a method for controlling humidity in a cell incubator, comprising the steps of providing a thermally conductive block having a water exuding surface in an interior of an incubator, controllably heating the block with a controllable electrical heater, blowing gas to be humidified over the surface of the block, sensing humidity at least downwind of the block with at least one humidity sensor, controllably pumping water to the exuding surface of the block and controlling the pumping of water to the surface of the block based upon an output from the at least one humidity sensor to obtain a desired level of humidity.

In some embodiments, the source of water is a water reservoir disposed exteriorly of the interior of the incubator.

The water exuding surface in some embodiments is wicking material attached to the block, grooves in a surface of the block, and/or a foam outer surface of the block.

The method in some embodiments includes a filtering the water between the source of water and the block and/or irradiating the water entering the block with a source of UV light.

The method in some embodiments includes using at least one temperature sensor at the block for sensing the temperature of the block and controlling the temperature of the block using electrical signals from the at least one temperature sensor to maintain a desired temperature of the block to prevent water from dripping from the exuding surface.

The method in some embodiments further comprising placing at least one humidity sensor upstream of the block and supplying a signal to the controller.

The method in some embodiments further comprises sterilizing the block by allowing the water exuding surface to dry and heating the block to a sterilizing temperature.

These and other features and advantages, which characterize the present non-limiting embodiments, will be apparent from a reading of the following detailed description and a review of the associated drawings. It is to be understood that both the foregoing general description and the following detailed description are explanatory only and are not restrictive of the non-limiting embodiments as claimed.

### BRIEF DESCRIPTION OF THE DRAWINGS

FIG. 1 is a perspective view of a humidity control device in accordance with the present invention;

FIG. 2 is another perspective view of the device of FIG. 1 with wicking material removed;

FIG. 3 is a perspective view of the device of FIG. 1 in a form mountable in an incubator;

FIG. 4 is a perspective view of a single culture flask incubator and imager with the device of FIG. 3 mounted therein;

FIG. 5 is a perspective view of a single culture plate incubator and imager with the device of FIG. 3 mounted therein;

FIG. 6 is a perspective view of an 8-slot culture flask incubator and imager with the device of FIG. 3 mounted therein;

FIG. 7 is a perspective view of an 8-slot culture plate incubator and imager with the device of FIG. 3 mounted therein; and

FIG. 8 is a block diagram of the controls for the humidity control device and incubator.

### DETAILED DESCRIPTION

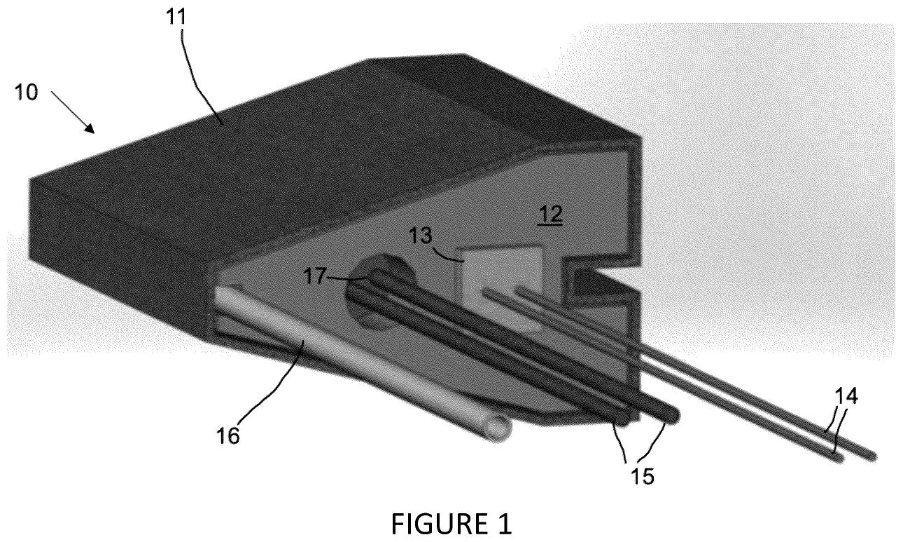

Referring to FIG. 1, the humidity control unit 10 includes a block 12 made of a thermally conductive material such as aluminum. In the embodiment shown, the block 12 is covered by cloth 11 which serves as a wicking material for the unit. Attached is block 12 is a temperature sensor 13 with wires 14 extending therefrom and providing a signal representative of the temperature that is sensed. Inserted into the block 12 is an electric heater 17 with wires 15 connected thereto and which provide a voltage and current to the heater such that the heater generates heat as a function thereof to heat the block to a desired temperature. Water is supplied to the unit 10 by a water tube 16 which wets the wicking material 11.

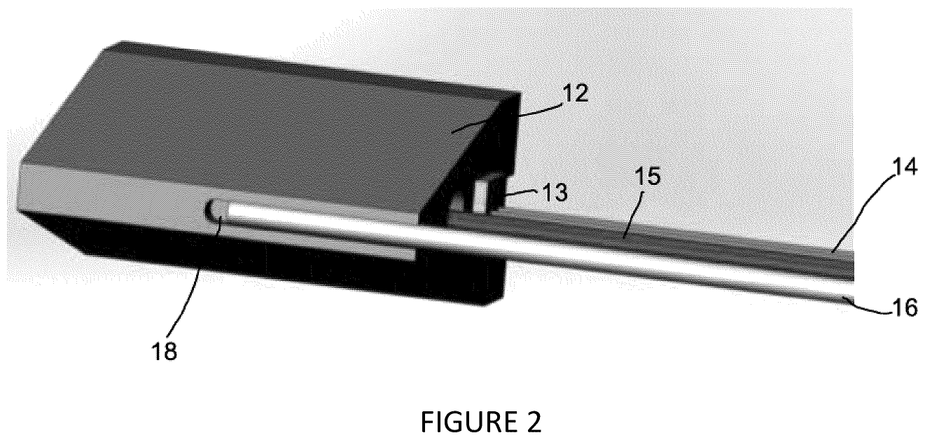

Referring to FIG. 2, the unit 10 is shown without the cover 11 so that one can see that tube 16 sits in a channel 18 so that water delivered by the tube will wet the wicking material 11 that covers block 12.

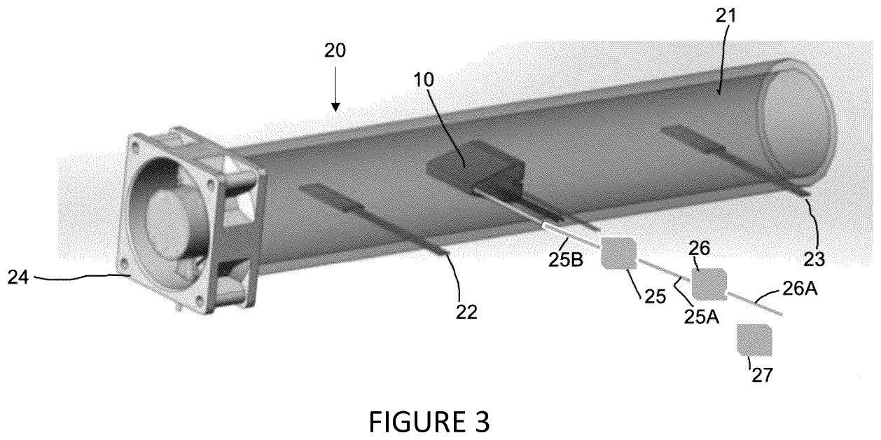

FIG. 3 shows an embodiment of the humidity control device 20 having the unit 10 in a tube or pipe 21 with a fan 24 at one end to blow a gas, such as oxygen past the unit 10. Also mounted in the tube 21 are humidity sensor 22 which is upstream of the unit 10 and humidity sensor 23 which is downstream of the unit 10. In the embodiment shown, the block 12 is wedge-shaped with the narrower or sharper end facing the fan to make the air flow more aerodynamic and to provide the most surface area of the block to the passing gas. Water is delivered to the tube 16 by pump 25 which has an outlet 25B connected to the tube and an inlet 25A which receives water from a reservoir. The device also includes a filter 26 and sterilizer 27 which is a source of UVC light controlled by the temperature controller that irradiates the water in inlet tube 26A delivered to the unit 10.

In other embodiments, the tube 26A is a glass tube and the sterilizer 27 is a heater controlled by the temperature controller which heats the water to a temperature sufficient to kill contaminating organisms in the water.

The apparatus of the present invention comprises the thermally conductive block, such as an aluminum block, covered with a wick, such as a piece of cotton cloth. The block is attached to an electrical heater and one or more temperature sensors. A fan blows the gas to be humidified over the cloth covered block and humidity sensors are placed either downwind or both upwind and downwind of the block. A pump, capable of delivering metered amounts of water, is connected via tubing between a reservoir of water and the block such that water pumped from the reservoir will wet the cloth. Water is pumped to the block under computer control, according to measurements from the temperature and humidity sensors. Thus the humidity can be set to any desired level.

It is desired that the amount of water pumped onto the cloth wick is not so much as to allow drops to fall or be blown off the wick. This can be controlled by noting the temperature of the block and the amount of power delivered to the heater to maintain that temperature. If the wick is wet, the heater's power will be expended turning water to vapor and the temperature of the block won't rise as much, or may fall. If the wick is dry, the power will go into raising the temperature of the block. Thus by monitoring the heater power and block temperature, the wetness of the wick can be evaluated and the supply of water to the wick can be controlled so it doesn't get overly wet and drip. By supplying the energy required to vaporize the water directly to the water, it doesn't have to come from the gas and the gas temperature remains constant.

It is important that the humidification system not introduce contaminants, especially viruses, bacteria, and fungi, into the incubator. To that end, the water going to the block can be filtered through a 0.2 μm filter to remove bacteria and fungi. Viruses can be eliminated by exposing the water to ultraviolet light as it travels from the pump to the block.

If it is desired to sterilize the block, the water pump can be turned off until the block is dry, then the fan can be turned off and sufficient power can be applied to the block to raise its temperature sufficiently to sterilize it.

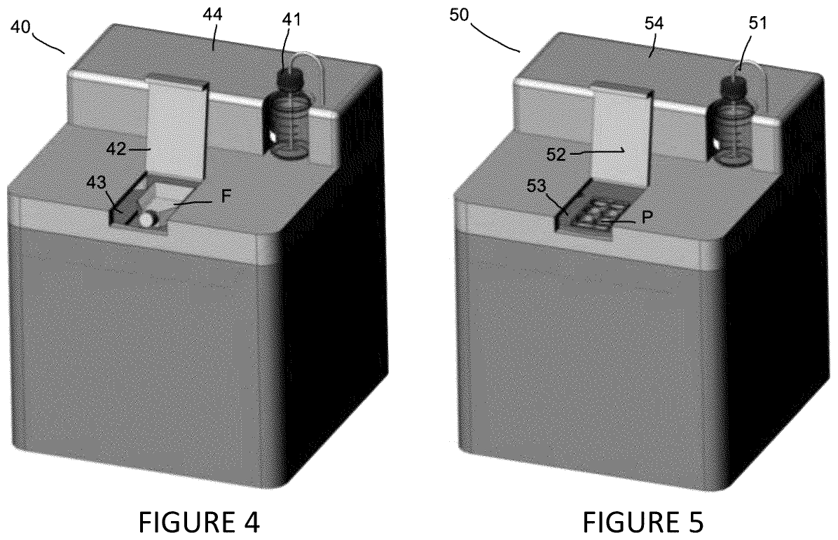

The water reservoir can be located outside the incubator so that the amount of water therein can be monitored, either manually or by computer, and the reservoir refilled without opening the incubator. This is an advantage over the standard method of simply placing a tray of water in the incubator. Examples of incubators with the device of FIG. 3 are shown in FIGS. 4-7.

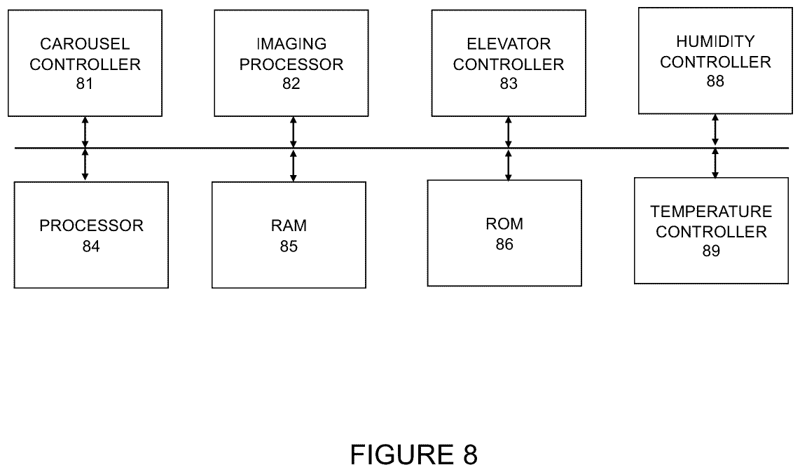

FIG. 4 shows an incubator 40 with a slot 43 with a lid 42 that is opened to accept a culture flask F. The water reservoir 41 is disposed exteriorly of the incubator 40 and the unit 20 is disposed in the interior of the incubator at a top portion 44. The incubator 40 also includes an imager in the interior thereof (not shown) and the flask F is lowered into the imager by an elevating mechanism (not shown). The humidity within the incubator is controlled by the device 20 and the control circuitry shown in FIG. 8.

FIG. 8 shows the basic elements of the incubator and imager. A main processor 84 controls the functions of the incubator and accesses RAM 85 and ROM 86 in which control software is stored. The imager is controlled by the imaging processor 82. The signals from the temperature sensor are received by temperature controller 89 and the signals form the humidity sensors are received by the humidity controller. The temperature controller controls the heater to heat the block to a desired temperature and the humidity controller controls the pump 26 to pump the appropriate amount of water to the unit 10 based upon the humidity and the temperature of the block. The raising and lowering of the elevator is controlled by the elevator controller 83. The controllers are microprocessors or microcontrollers configured to perform the desired functions.

FIG. 5 shows an incubator 50 with a slot 53 opened with lid 52 and receptive of a culture plate P. The device 20 is under cover 54 and is supplied by water reservoir 51. The operation of incubator 50 is similar to that of incubator 40.

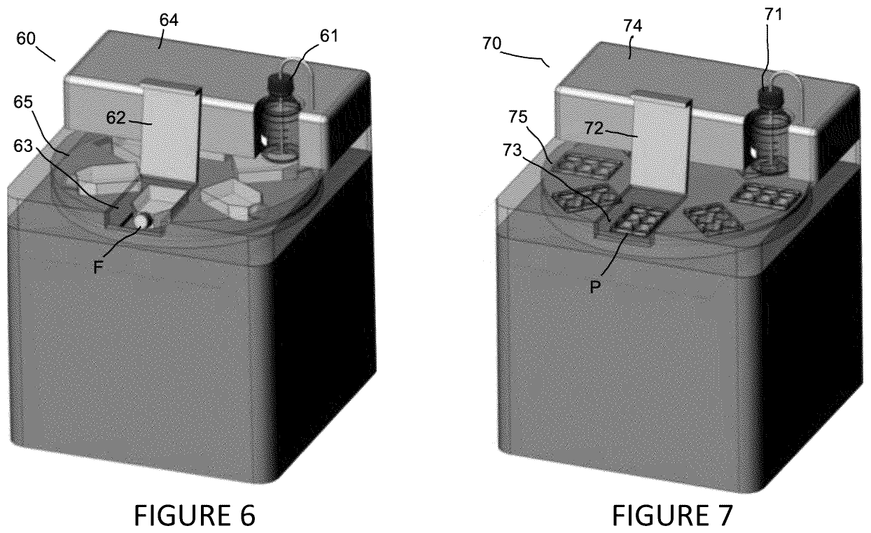

FIGS. 6 and 7 show variants of the incubators of FIGS. 4 and 5. These incubators 60 and 70 include carousels 65 and 75 which receive up to 8 culture flasks F or 8 culture plates P respectively through slots 63, 73 opened by doors 62, 72. As in the other embodiments, the devices 20 are disposed under in the interior of the incubator under covers 64, 74 and are supplied by exteriorly mounted reservoirs 61, 71. In these embodiments, the imagers within the incubators are able to image any of the 8 flasks or plates by controlling the carousel using carousel controller 81 in conjunction with the elevator controller and imaging processor.

The cotton cloth wick could be any wettable fabric, comprised of natural and/or synthetic fibers. It could also be a foam-like structure like a sponge. Instead of a wick, small grooves could be machined into the block to retain water via capillary action. Alternately, the entire block could be machined out a foam-like structure comprised of a material with suitable thermal conductivity and wettability, thus obviating the need for a wick.

The shape of the block can be different from that shown. For example, it can be rectangular or other regular or irregular solid shape. The size of the block and in particular the surface area thereof, can be changed to be larger or smaller depending upon the amount of air to be humidified. The block is generally a rectangular solid shape although other shapes are within the scope of the invention. The edges can be rounded or sharp. The block can be made out of metal or thermally conductive plastic. The wicking material can be a cloth or a fabric which is either natural or synthetic.

While a fan is shown, other gas moving devices can be used instead of or in addition to a fan. While UVC light is mentioned as a source of sterilization, other sources of light can be used and it is understood that UVC may be used when it is necessary to protect against viruses.

An elevating mechanism for use moving a flask or a culture plate from the input slot in the incubator in either the single flask or plate embodiment or in the rotating carousel 8 slot flask or culture plate embodiment is disclosed in application Ser. No. 15/563,370 filed on Sep. 29, 2017, the disclosure of which is hereby incorporated by reference herein.

In other embodiments the invention can be used with vessels other than cell culture plates and flasks, with optical instruments other than imagers.

The various methods or processes outlined herein may be coded as software that is executable on one or more processors that employ any one of a variety of operating systems or platforms. Such software may be written using any of a number of suitable programming languages and/or programming or scripting tools and may be compiled as executable machine language code or intermediate code that is executed on a framework or virtual machine.

One or more algorithms for controlling methods or processes provided herein may be embodied as a readable storage medium (or multiple readable media) (e.g., a non-volatile computer memory, one or more floppy discs, compact discs (CD), optical discs, digital versatile disks (DVD), magnetic tapes, flash memories, circuit configurations in Field Programmable Gate Arrays or other semiconductor devices, or other tangible storage medium) encoded with one or more programs that, when executed on one or more computing units or other processors, perform methods that implement the various methods or processes described herein.

In various embodiments, a computer readable storage medium may retain information for a sufficient time to provide computer-executable instructions in a non-transitory form. Such a computer readable storage medium or media can be transportable, such that the program or programs stored thereon can be loaded onto one or more different computing units or other processors to implement various aspects of the methods or processes described herein. As used herein, the term “computer-readable storage medium” encompasses only a computer-readable medium that can be considered to be a manufacture (e.g., article of manufacture) or a machine. Alternately or additionally, methods or processes described herein may be embodied as a computer readable medium other than a computer-readable storage medium, such as a propagating signal.

The terms “program” or “software” are used herein in a generic sense to refer to any type of code or set of executable instructions that can be employed to program a computing unit or other processor to implement various aspects of the methods or processes described herein. Additionally, it should be appreciated that according to one aspect of this embodiment, one or more programs that when executed perform a method or process described herein need not reside on a single computing unit or processor but may be distributed in a modular fashion amongst a number of different computing units or processors to implement various procedures or operations.

Executable instructions may be in many forms, such as program modules, executed by one or more computing units or other devices. Generally, program modules include routines, programs, objects, components, data structures, etc., that perform particular tasks or implement particular abstract data types. Typically, the functionality of the program modules may be organized as desired in various embodiments.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, e.g., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, e.g., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (e.g. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, e.g., to mean including but not limited to.

Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A device insertable into an interior of an incubator for controlling humidity in the incubator, comprising:
a thermally conductive block having a water exuding surface;
an electrical heater attached to the block and receptive of electrical energy to heat the block;
at least one fan for blowing gas to be humidified over the surface of the block;
at least humidity sensor attached at least downwind of the block;
a controllable pump connectable to a source of water for supplying water to the exuding surface of the block; and
a controller receptive of output signals from the at least one humidity sensor and connected to the pump for controlling the pump based upon the output signals from the at least one humidity sensor to obtain a desired level of humidity.

2. The device according to claim 1, wherein the water exuding surface is wicking material attached to the block.

3. The device according to claim 1, wherein the water exuding surface are grooves in a surface of the block.

4. The device according to claim 1, wherein the water exuding surface is a foam outer surface of the block.

5. The device according to claim 1, further comprising a filter between the source of water and the block.

6. The device according to claim 1, further comprising a source of UV light irradiating water entering the block.

7. The device according to claim 1, further comprising at least one temperature sensor at the block for sensing the temperature of the block and wherein the controller is receptive of electrical signals from the at least one temperature sensor to maintain a desired temperature of the block to prevent water from dripping from the exuding surface.

8. The device according to claim 1, further comprising at least one humidity sensor upstream of the block and supplying a signal to the controller.

9. An apparatus for controlling humidity in a cell incubator, comprising:
A cell incubator having an interior;
a thermally conductive block having a water exuding surface disposed in the interior of the incubator;
an electrical heater attached to the block and receptive of electrical energy to heat the block;
at least one fan for blowing gas to be humidified over the surface of the block;
at least humidity sensor attached at least downwind of the block;
a controllable pump connectable to a source of water for supplying water to the exuding surface of the block; and
a controller receptive of output signals from the at least one humidity sensor and connected to the pump for controlling the pump based upon the output signals from the at least one humidity sensor to obtain a desired level of humidity.

10. The apparatus according to claim 9, wherein the source of water is a water reservoir disposed exteriorly of the interior of the incubator.

11. The apparatus according to claim 9, wherein the water exuding surface is wicking material attached to the block.

12. The apparatus according to claim 9, wherein the water exuding surface are grooves in a surface of the block.

13. The apparatus according to claim 9, wherein the water exuding surface is a foam outer surface of the block.

14. The apparatus according to claim 9, further comprising a filter between the source of water and the block.

15. The apparatus according to claim 9, further comprising a source of UV light irradiating water entering the block.

16. The apparatus according to claim 9, further comprising at least one temperature sensor at the block for sensing the temperature of the block and wherein the controller is receptive of electrical signals from the at least one temperature sensor to maintain a desired temperature of the block to prevent water from dripping from the exuding surface.

17. The apparatus according to claim 9, further comprising at least one humidity sensor upstream of the block and supplying a signal to the controller.

18. A method for controlling humidity in a cell incubator, comprising the steps of:
providing a thermally conductive block having a water exuding surface in an interior of an incubator;
controllably heating the block with a controllable electrical heater;
blowing gas to be humidified over the surface of the block;
sensing humidity at least downwind of the block with at least one humidity sensor;
controllably pumping water to the exuding surface of the block; and
controlling the pumping of water to the surface of the block based upon an output from the at least one humidity sensor to obtain a desired level of humidity.

19. The method according to claim 18, wherein the water is in a reservoir disposed exteriorly of the interior of the incubator.

20. The method according to claim 18, wherein the water exuding surface is wicking material attached to the block.

21. The method according to claim 18, wherein the water exuding surface comprises grooves in a surface of the block.

22. The method according to claim 18, wherein the water exuding surface is a foam outer surface of the block.

23. The method according to claim 18, further comprising filtering the water to the block.

24. The method according to claim 18, further comprising irradiating water entering the block with UV light.

25. The method according to claim 18, further comprising sensing the temperature of the block with at least one temperature sensor and wherein the step of controlling comprises receiving an input the at least one temperature sensor and maintaining a desired temperature of the block to prevent water from dripping from the exuding surface.

26. The method according to claim 18, further comprising placing at least one humidity sensor upstream of the block and supplying a signal to the controller.

27. The method according to claim 18, further comprising sterilizing the block by allowing the water exuding surface to dry and heating the block to a sterilizing temperature.
